(19) United States
(12) Patent Application Publication
(10) Pub. No.: US 2026/0207063 A1
(43) Pub. Date: Jul. 23, 2026

(54) Digital Pressure Transducer with Message Integrity Validation Capability

(71) Applicant: Becton, Dickinson and Company, Franklin Lakes, NJ (US)

(72) Inventors: Oleg Yusim, Franklin Lakes, NJ (US); Donald Ray Myers, Franklin Lakes, NJ (US); Sudarsan Pranatharthikaran, Franklin Lakes, NJ (US); Catherine M. Szyman, Franklin Lakes, NJ (US); Ryan Timothy McHale, Franklin Lakes, NJ (US); Shaun Fetherston, Franklin Lakes, NJ (US)

(21) Appl. No.: 19/139,242

(22) PCT Filed: Dec. 19, 2023
(86) PCT No.: PCT/US2023/084939

§ 371 Date: Jun. 13, 2025

Related U.S. Application Data
(60) Provisional application No. 63/476,367, filed on Dec. 20, 2022.

(51) Int. Cl. A61B 5/0215; A61B 5/00

(52) U.S. Cl. CPC A61B 5/0215; A61B 5/7221; A61B 5/7225; A61B 5/746

(57) ABSTRACT

Disclosed is a digital pressure transducer for use in a blood pressure monitoring system to measure the blood pressure of a patient and to provide integrity validation. The blood pressure monitoring system includes a measurement site, a tube, and a host device. The tube is coupled between the measurement site of the patient and the digital pressure transducer and contains fluid. The digital pressure transducer comprises: an analog pressure transducer to measure an analog pressure of a fluid pressure from the fluid of the tube from the measurement site; an ADC to convert the analog pressure measurement to a digital pressure measurement; an interface to connect to the host device; and a processor configured to: convert the digital pressure measurement to a digital blood pressure measurement for the patient; transmit the digital blood pressure measurement of the patient to the host device; and perform integrity validation with the host device.

### CROSS-REFERENCE TO RELATED APPLICATIONS

This application claims the benefit of U.S. Provisional Ser. No. 63/476,367 filed Dec. 20, 2022, which is incorporated by reference herein in its entirety.

### FIELD

The invention relates to a digital pressure transducer with message integrity validation capability.

### BACKGROUND

### Relevant Background

Blood pressure measurement systems often utilize pressure transducers (PTs) that are used to monitor blood pressure signals in a patient's vein or artery. PTs can also be used to monitor intracranial pressure as well as a wide variety of other types of pressure measurements. In particular, electrical pressure signals generated by PTs may be used for a number of monitoring and diagnostic applications and are often connected to a patient monitor to display graphical depictions of the signals generated, such as, pressure vs. time, etc. A PT is typically mounted near the patient and connected to the patient's vein, artery, cranium, or other part of the body via a catheter and a fluid-filled tube and to the patient monitor. Oftentimes, the PTs may be disposable pressure transducers (DPTs)). Patient monitors may employ sophisticated algorithms to derive volumetric and hemodynamic parameters from the pressure signal.

In particular, the pressure signal is generated and transmitted from the measurement site (e.g., vein, artery, etc.) via the catheter and the fluid-filled tube as fluid pressure to the pressure transducer (PT) where it may be converted to an electrical pressure signal outputted to the patient monitor. The PT is typically located in a plastic enclosure that ensures connectivity to the fluid-filled catheter-tubing system on one side and the patient monitor on the other side. The term PT or DPT usually refers to the system that includes the enclosure housing the pressure transducer, the transducer itself, and respective connectors. Therefore, as has been described, in these type of pressure measurement systems, a pressure transducer may be connected to a patient's artery or vein through a fluid column contained in tubing on one side and may output electrical pressure signals from the PT on the other side to the patient monitor.

In the current implementations, these types of pressure transducers (PTs) are analog and they do not have the capability to uniquely identify themselves to a patient monitor. Therefore, there is no way for a patient monitor to authenticate a PT and be sure that the PT is a legitimate PT for use by the patient monitor and that its data can be trusted. Further, there is no way for the PT to provide an integrity validation process to the patient monitor to validate the integrity of its pressure reading data to ensure that it has not been interfered with, manipulated, or tampered with.

### SUMMARY

Embodiments relate to a digital pressure transducer with message integrity validation capability. In particular, embodiments are related to a digital pressure transducer that can provide a message integrity validation process to a host device to validate the integrity of its pressure reading data to ensure that it has not been interfered with, manipulated, or tampered with.

In one embodiment, disclosed is a digital pressure transducer for use in a blood pressure monitoring system to measure the blood pressure of a patient and to provide integrity validation. The blood pressure monitoring system may include a measurement site, a tube, and a host device, in which, the tube is coupled between the measurement site of the patient and the digital pressure transducer, and, in which, the tube contains a fluid for pressure measurement from the measurement site. The digital pressure transducer may comprise: an analog pressure transducer to measure an analog pressure of a fluid pressure from the fluid of the tube from the measurement site; an analog to digital converter (ADC) to convert the analog pressure measurement to a digital pressure measurement; an interface to connect to the host device; and a processor. The processor may be configured to: convert the digital pressure measurement to a digital blood pressure measurement for the patient; transmit the digital blood pressure measurement of the patient to the host device; and perform integrity validation with the host device.

In one optional example embodiment, the processor may further be configured to perform integrity validation for a predefined number of digital blood pressure measurements transmitted to the host device based upon a hash-based message authentication code (HMAC). In another optional example embodiment, the digital pressure transducer further includes a secret key. In an additional optional example embodiment, to perform integrity validation with the host device, the processor is further configured to: obtain a predefined number of digital blood pressure measurements transmitted to the host device; generate a hash-based message authentication code (HMAC) based upon the predefined number of digital blood pressure measurements and the secret key; and command the interface to transmit the HMAC to the host device. In one optional example embodiment, the interface is a digital cable, and the digital cable may be connected to the host device, and the digital cable may transmit integrity validation data for the performing of integrity validation with the host device and the digital blood pressure measurement of the patient to the host device. In a further optional example embodiment, the interface may be a wireless transceiver that wirelessly connects over the air to a transceiver of the host device, and the transceiver wirelessly transmits and receives integrity validation data for the performing of integrity validation with the host device and wirelessly transmits the digital blood pressure measurement of the patient to the host device. In another optional example embodiment, the interface is a digital cable, and the digital cable may be connected to the host device, and the digital cable performs integrity validation with the digital pressure transducer, and the digital cable transmits the digital blood pressure measurement of the patient to the host device. In one optional example embodiment, the digital pressure transducer is a disposable pressure transducer.

It should be appreciated that the optional examples may be utilized independently from one another or in combination with one another.

In one embodiment, disclosed is a non-transitory computer-readable medium comprising code which, when executed by a processor of digital pressure transducer for use in a blood pressure monitoring system is used to measure the blood pressure of a patient and to provide integrity validation. The blood pressure monitoring system includes a measurement site, a tube, and a host device, the tube coupled between the measurement site of the patient and the digital pressure transducer, and the tube containing a fluid for pressure measurement from the measurement site. The processor is caused by execution of the code to perform operations comprising: converting a digital pressure measurement to a digital blood pressure measurement for the patient, wherein the digital pressure measurement is received from an analog to digital converter (ADC) that converts an analog pressure measurement of a fluid pressure from the fluid of the tube from the measurement site to the digital pressure measurement; transmitting the digital blood pressure measurement of the patient to the host device; and performing integrity validation with the host device.

In one optional example embodiment, to perform integrity validation with the host device, the processor further performs the operation of perform integrity validation for a predefined number of digital blood pressure measurements transmitted to the host device based upon a hash-based message authentication code (HMAC). In another optional example embodiment, the digital pressure transducer further includes a secret key. In an additional optional example embodiment, to perform integrity validation with the host device, the processor further performs the operations of: obtaining a predefined number of digital blood pressure measurements transmitted to the host device; generating a hash-based message authentication code (HMAC) based upon the predefined number of digital blood pressure measurements and the secret key; and commanding the interface to transmit the HMAC to the host device. In one optional example embodiment, the interface is a digital cable, and the digital cable may be connected to the host device, and the digital cable may transmit integrity validation data for the performing of integrity validation with the host device and the digital blood pressure measurement of the patient to the host device. In a further optional example embodiment, the interface may be a wireless transceiver that wirelessly connects over the air to a transceiver of the host device, and the transceiver wirelessly transmits and receives integrity validation data for the performing of integrity validation with the host device and wirelessly transmits the digital blood pressure measurement of the patient to the host device. In another optional example embodiment, the interface is a digital cable, and the digital cable may be connected to the host device, and the digital cable performs integrity validation with the digital pressure transducer, and the digital cable transmits the digital blood pressure measurement of the patient to the host device. In one optional example embodiment, the digital pressure transducer is a disposable pressure transducer.

It should be appreciated that the optional examples may be utilized independently from one another or in combination with one another.

In one embodiment, disclosed is a blood pressure monitoring system to measure the blood pressure of a patient from a measuring site and to provide integrity validation. The blood pressure monitoring system may include a host device, a tube coupled between the measurement site of the patient and a digital pressure transducer, and, in which, the tube contains a fluid for pressure measurement from the measurement site. The digital pressure transducer may comprise: an analog pressure transducer to measure an analog pressure of a fluid pressure from the fluid of the tube from the measurement site; an analog to digital converter (ADC) to convert the analog pressure measurement to a digital pressure measurement; an interface to connect to the host device; and a processor. The processor may be configured to: convert the digital pressure measurement to a digital blood pressure measurement for the patient; transmit the digital blood pressure measurement of the patient to the host device; and perform integrity validation with the host device.

In one optional example embodiment, the processor may further be configured to perform integrity validation for a predefined number of digital blood pressure measurements transmitted to the host device based upon a hash-based message authentication code (HMAC). In another optional example embodiment, the digital pressure transducer further includes a secret key. In an additional optional example embodiment, to perform integrity validation with the host device, the processor is further configured to: obtain a predefined number of digital blood pressure measurements transmitted to the host device; generate a hash-based message authentication code (HMAC) based upon the predefined number of digital blood pressure measurements and the secret key; and command the interface to transmit the HMAC to the host device. In one optional example embodiment, the interface is a digital cable, and the digital cable may be connected to the host device, and the digital cable may transmit integrity validation data for the performing of integrity validation with the host device and the digital blood pressure measurement of the patient to the host device. In a further optional example embodiment, the interface may be a wireless transceiver that wirelessly connects over the air to a transceiver of the host device, and the transceiver wirelessly transmits and receives integrity validation data for the performing of integrity validation with the host device and wirelessly transmits the digital blood pressure measurement of the patient to the host device. In another optional example embodiment, the interface is a digital cable, and the digital cable may be connected to the host device, and the digital cable performs integrity validation with the digital pressure transducer, and the digital cable transmits the digital blood pressure measurement of the patient to the host device. In one optional example embodiment, the digital pressure transducer is a disposable pressure transducer.

It should be appreciated that the optional examples may be utilized independently from one another or in combination with one another.

In one embodiment, disclosed is a digital pressure transducer for use in a blood pressure monitoring system to measure the blood pressure of a patient and to provide integrity validation. The blood pressure monitoring system may include a measurement site, a tube, and a host device, in which, the tube is coupled between the measurement site of the patient and the digital pressure transducer, and the tube contains a fluid for pressure measurement from the measurement site. The digital pressure transducer may comprise: an analog pressure transducer to measure an analog pressure of a fluid pressure from the fluid of the tube from the measurement site; an analog to digital converter (ADC) to convert the analog pressure measurement to a digital pressure measurement; a secret key; an interface to connect to the host device; and a processor. The processor may be configured to perform integrity validation with the host device by performing operations including: obtaining a predefined number of digital blood pressure measurements previously transmitted to the host device; generating a hash-based message authentication code (HMAC) based upon the predefined number of digital blood pressure measurements and the secret key; commanding the interface to transmit the HMAC to the host device such that the host device is capable of integrity validation of the predefined number of digital blood pressure measurements; converting subsequent digital pressure measurements to digital blood pressure measurements for the patient; and transmitting the subsequent digital blood pressure measurements of the patient to the host device.

### BRIEF DESCRIPTION OF THE DRAWINGS

FIG. 1 is a diagram illustrating an example environment in which embodiments of the invention may be practiced.

FIG. 2 is a diagram illustrating an embodiment of a digital pressure transducer for use in a blood pressure monitoring system connected to a host device via a digital cable, according to embodiments of the invention.

FIG. 3 is a diagram illustrating an embodiment of a digital pressure transducer for use in a blood pressure monitoring system connected to a host device wirelessly, according to embodiments of the invention.

FIG. 4 is a diagram illustrating an embodiment of a digital pressure transducer with more detailed components, according to embodiments of the invention.

FIG. 5 is a flow diagram illustrating an authentication process for the digital pressure transducer, according to embodiments of the invention.

FIG. 6 is a flow diagram illustrating an integrity validation process for the digital pressure transducer, according to embodiments of the invention.

### DETAILED DESCRIPTION

Various embodiments and optional examples of the disclosures will be described with reference to details discussed below, and the accompanying drawings will illustrate the various optional examples and embodiments. The following description and drawings are illustrative of the disclosure and are not to be construed as limiting the disclosure. Numerous specific details are described to provide a thorough understanding of various embodiments and optional examples of the disclosure. However, in certain instances, well-known or conventional details are not described in order to provide a concise discussion of embodiments and optional examples of the disclosures. It should be appreciated that the terms “embodiment” or “example” or “optional example” or “optional example embodiment” may be considered to have the same meaning and may be used interchangeably with one another. Reference in the specification to “embodiment” or “example” or “optional example” or “optional example embodiment” means that a particular feature, structure, or characteristic described in conjunction with the “embodiment” or “example” or “optional example” or “optional example embodiment” may be included.

Embodiments relate to a digital pressure transducer with message integrity validation capability for use in a blood pressure monitoring system that can validate the integrity of its digital blood pressure readings to the host device, such that the host device can be assured that the digital blood pressure readings that it is receiving from the digital pressure transducer have not been interfered with, manipulated, or tampered with.

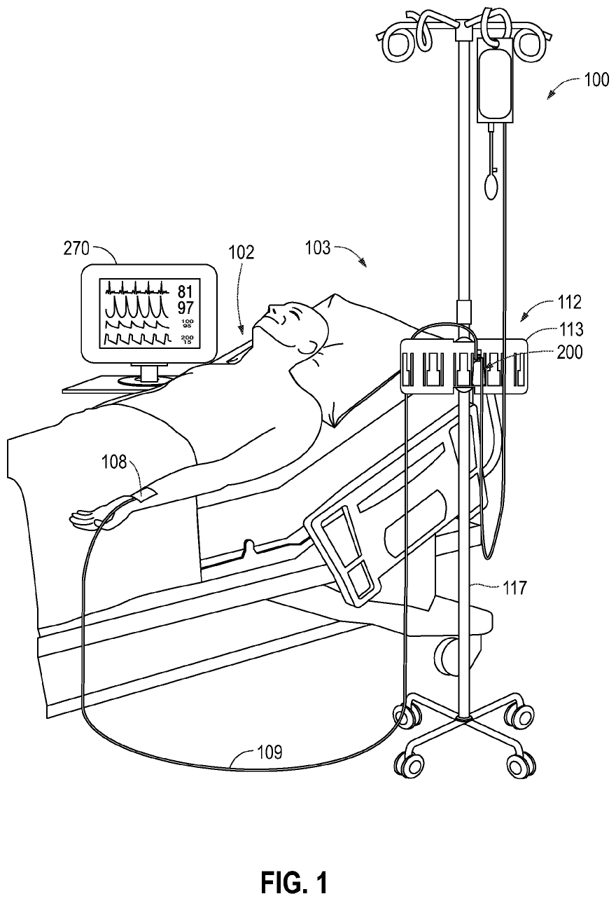

FIG. 1 is a diagram illustrating an example environment 100 in which embodiments of the invention may be practiced. In this example, a pressure monitoring system 103 for a patient 102 may be utilized that includes a measurement site 108, a tube 109, and a digital pressure transducer 200, in which, the tube 109 is coupled between the measurement site 108 of the patient 102 and the digital pressure transducer 200. Patient 102 may be, as an example, lying on a bed.

In this example environment 100, the blood pressure measurement system may utilize digital pressure transducer 200 to monitor the blood pressure signals in the patient's 102 vein or artery. As one example, digital blood pressure signals generated by the digital pressure transducer 200 may be used for a number of monitoring and diagnostic applications and may be connected to a patient monitor or host device 270 to display graphical depictions of the signals generated, such as, pressure vs. time, etc. As can be seen an example of host device 270 such items as blood pressure, heart rate, etc., may be displayed.

A catheter may be connected to patient's 102 vein or artery at measurement site 108. It should be appreciated that this is just an example of measurement site at a patient's wrist and the measurement site 108 may be at any suitable patient location. Further, a pressure signal is generated and transmitted from the measurement site 108 (e.g., vein, artery, etc.) via the catheter and the fluid-filled tube 109 as fluid pressure to the pressure transducer the digital pressure transducer 200 where it may be converted to an electrical pressure signal, and then may be converted into a digital pressure signal, and then further may be converted to a digital blood pressure measurement for the patient and may be outputted to the host device 270. Therefore, digital pressure transducer 200 may be connected to a patient's artery or vein at measurement site 108 through a fluid column contained in the tubing 109 on one side and may output digital blood pressure measurements for the patient from the digital pressure transducer 200 on the other side to host device 270 for display. The digital pressure transducer 200 may be a digital disposable pressure transducer (DPT).

As shown in this example environment 100, a digital pressure transducer 200 may be connected to a plate 113 of a sensor holding apparatus 112 that is designed for holding various different types of transducers, sensors, medical devices, etc. As can be in seen FIG. 1, sensor holding apparatus 112 may be approximately rectangular cuboid shaped including six sides and may have a front plate 113 that may have various sensor holders for holding various types of sensors, transducers, medical devices, etc. Sensors that may be placed into the sensor holders may include, for example, digital pressure transducer 200. Digital pressure transducer 200 may be considered to be a system that includes the enclosure housing for the pressure transducer, the pressure transducer itself, an analog to digital converter (ADC), a processor, associated memory and circuitry, respective connectors, as will be described in more detail hereafter.

In this example environment 100, the sensor holding apparatus 112 for holding digital pressure transducer 200, as well, as other types of sensors, transducers, medical devices, etc., may be mounted to a rolling IV stand 117 that further may be used to mount an IV bag mounted on the top of the stand to provide fluids. In particular, it should be appreciated that digital pressure transducer 200 may be located in a plastic enclosure that is mounted to a sensor holder of the sensor holding apparatus 112 that ensures connectivity to the fluid-filled tube 109 and catheter measurement site 108 on one side and the host device 270 on the other side. In this way, digital pressure transducer 200 may be connected to a patient's artery or vein through a fluid column contained in tubing 109 on one side and may output digital blood pressure signals from digital pressure transducer 200 on the other side to the host device 270 for display. A connection (e.g., a cable) is not shown between digital pressure transducer 200 and host device 270 in this example environment. As will be described hereafter, different types of wired cable and/or wireless connections may be utilized between digital pressure transducer 200 and host device 270.

It should be appreciated that a wide variety of other types of sensors, transducers, medical devices, etc., may be mounted to the sensor holders of sensor holding apparatus 112, such as, various other types of DPTs that may be for the measuring of Pulmonary Artery Pressure (PAP), Central Venous Pressure (CVP), Arterial Pressure (AP), etc.

However, it should likewise be appreciated that a sensor holding apparatus 112 may not be utilized at all, and in some optional example embodiments, digital pressure transducer 200 may be connected to patient 102 by other implementations, such as being mounted to the patient directly or to other types mounting structures, and the digital pressure transducer 200 being wirelessly connected or connected physically (e.g., via a cable) to host device 270.

By utilizing this example environment, digital pressure transducer 200 by measuring the pressure differences of the fluid in tube 109 may generate analog electrical pressure signals, which may be converted into digital pressure signals, and then further may be converted into digital blood pressure measurements corresponding the patient's blood pressure as measured at measurement site 108 and may be outputted to the host device 270 for display and/or further analysis. As an example, the digital blood pressure measurements may be used for a number of monitoring diagnostic applications and, in particular, may be displayed on host device 270 to display a graphical depiction of blood pressure vs. time, as well as other types of data. It should be appreciated that a wide variety of other types of physiological data may be measured and displayed utilizing a suitable medical sensor device located at the sensor holding apparatus 112, and these are merely examples.

In one embodiment, disclosed is a digital pressure transducer 200 for use in a blood pressure monitoring system to measure the blood pressure of a patient 102 and to provide authentication. The blood pressure monitoring system may include a measurement site 108, a tube 109, and a host device 270, in which, the tube 109 is coupled between the measurement site 108 of the patient 102 and the digital pressure transducer 200. As has been described, tube 109 contains a fluid for pressure measurement from the measurement site. Digital pressure transducer 200 may comprise: an analog pressure transducer to measure an analog pressure of a fluid pressure from the fluid of the tube 109 from the measurement site; an analog to digital converter (ADC) to convert the analog pressure measurement to a digital pressure measurement; an interface to connect to the host device 270; and a processor. The processor may be configured to: perform authentication with the host device 270; convert the digital pressure measurement to a digital blood pressure measurement for the patient 210; and transmit the digital blood pressure measurement of the patient to the host device 270. As an example, the digital blood pressure measurement may be displayed on the host device 270 and/or may be used for a number of monitoring diagnostic applications. More details of the digital pressure transducer 200 will be described hereafter.

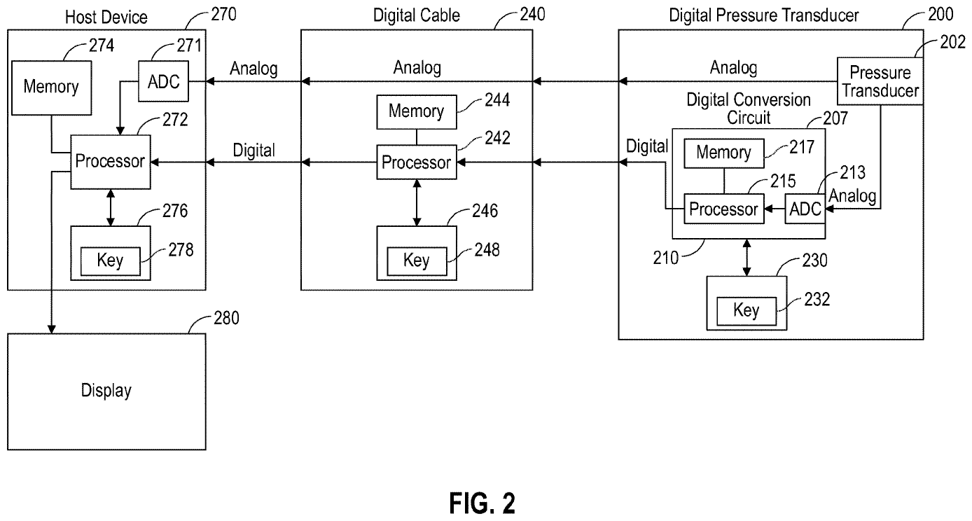

With additional reference to FIG. 2, disclosed is an embodiment of a digital pressure transducer 200 for use in a blood pressure monitoring system to measure the blood pressure of a patient and to provide authentication. As previously described with reference to FIG. 1, the blood pressure monitoring system may include a measurement site 108, a tube 109, and a host device 270, in which, the tube 109 is coupled between the measurement site 108 of the patient 102 and the digital pressure transducer 200, and, in which, the tube 109 contains a fluid for pressure measurement from the measurement site 108. In particular, as shown FIG. 2, the digital pressure transducer 200 may comprise: an analog pressure transducer 202 to measure an analog pressure of a fluid pressure from the fluid of the tube from the measurement site; an analog to digital converter (ADC) 213 to convert the analog pressure measurement from the analog pressure transducer to a digital pressure measurement; an interface to connect to the host device 270; and a processor 215. As will be described, processor 215 may be configured to: perform authentication with the host device 270; convert the digital pressure measurement to a digital blood pressure measurement for the patient; and transmit the digital blood pressure measurement of the patient to the host device 270. In one example, digital pressure transducer 200 and host device 270 determine the authenticity of each other to start communication and thereafter the digital pressure transducer 200 transmits the digital blood pressure measurements of the patient to the host device 270 through an interface.

In one optional example embodiment, as shown in FIG. 2, the digital pressure transducer 200 includes: an analog pressure transducer 202, a digital conversion circuit 207, and a protected memory 230. The protected memory 230 stores a secret key 232. The digital conversion circuit 207 includes: an analog to digital converter (ADC) 213, a processor 215, and a memory 217. As has been described, the analog pressure transducer 202 measures an analog pressure of a fluid pressure from the fluid of the tube from the measurement site. The analog pressure signal is transmitted to ADC 213 to convert the analog pressure measurement from the analog pressure transducer 202 to a digital pressure measurement. Processor 215 is configured to convert the digital pressure measurement to a digital blood pressure measurement that is correlated to the blood pressure of the patient and to transmit the digital blood pressure measurement of the patient to the host device 270 through an interface. It should be appreciated that the digital blood pressure measurement is a “continuous” blood pressure measurement. In one optional example embodiment, as shown in FIG. 2, and as will be described in more detail hereafter, the interface is a digital cable 240. Also, as will be described in more detail hereafter, the processor 210 may utilize the secret key for authentication purposes. Additionally, in some optional example embodiments, the analog pressure signal from the pressure transducer 202 may be directly connected the host device 270. It should be appreciated that the optional examples may be utilized independently from one another or in combination with one another.

Further, in one optional embodiment example, as will be described in more detail, digital pressure transducer 200 has authentication capabilities to authenticate itself to host device 270, such that the host device 270 can be assured that it is receiving data from a legitimate digital pressure transducer. In this way, the digital pressure transducer 200 and the host device 270 determine the authenticity of each other to start communication and thereafter the digital pressure transducer 200 transmits the digital blood pressure measurements of the patient to the host device 270 through an interface. It should be appreciated that the optional examples may be utilized independently from one another or in combination with one another.

In the optional example embodiment, where the digital pressure transducer is connected through a digital cable 240 to the host device 270, the digital cable 240 will be described. When a digital cable 240 is utilized, digital cable 240 transmits the digital blood pressure measurement of the patient from digital pressure transducer 200 to host device 270 and also transmits authentication data for performing authentication of pressure transducer 200 with host device 270, as will be described in more detail hereafter. Also, in some optional example embodiments, the digital cable 240 may also transmit the analog pressure signal from the pressure transducer 202 to the host device. In some optional example embodiments, the digital cable 240 includes a secure memory 246 storing a secret key 248 that may perform authentication functions, as will be described in more detail hereafter.

Continuing with this optional example embodiment of utilizing a digital cable 240 to connect digital pressure transducer 200 to host device 270, host device 270 may include: a processor 272, a memory 274, a secure memory 276 storing a secret key 278, an ADC 271 and may be connected to a display 280. In this optional example embodiment, assuming authentication has successfully occurred, host device 270 under the control of processor 272 may receive the digital blood pressure measurements from the digital pressure transducer 240 through the digital cable 240 corresponding to the patient's blood pressure and may utilize them in monitoring diagnostic applications and/or other applications, and/or, may display the digital blood pressure measurements on display 280 (e.g., as a graphical depiction of blood pressure vs. time). It should be appreciated that a wide variety of other types of physiological data may be measured and displayed.

Further, in some optional example embodiments, the host device 270 may convert the received analog pressure signals from digital cable 240 from digital pressure transducer 200 and via ADC 271 convert the analog pressure signal into digital pressure signals for processing by processor 272 such that the patient's digital blood pressure measurements can be analyzed and displayed, as previously described. Also, in some optional example embodiments, the secret key 278 of secure memory 276 may be used with the digital pressure transducer 200 for authentication functions, as will be described in more detail hereafter. As has been described, in one example, digital pressure transducer 200 and host device 270 may determine the authenticity of each other to start communication and thereafter the digital pressure transducer 200 transmits the digital blood pressure measurements of the patient to the host device 270 through the digital cable 240. It should be appreciated that the optional examples may be utilized independently from one another or in combination with one another.

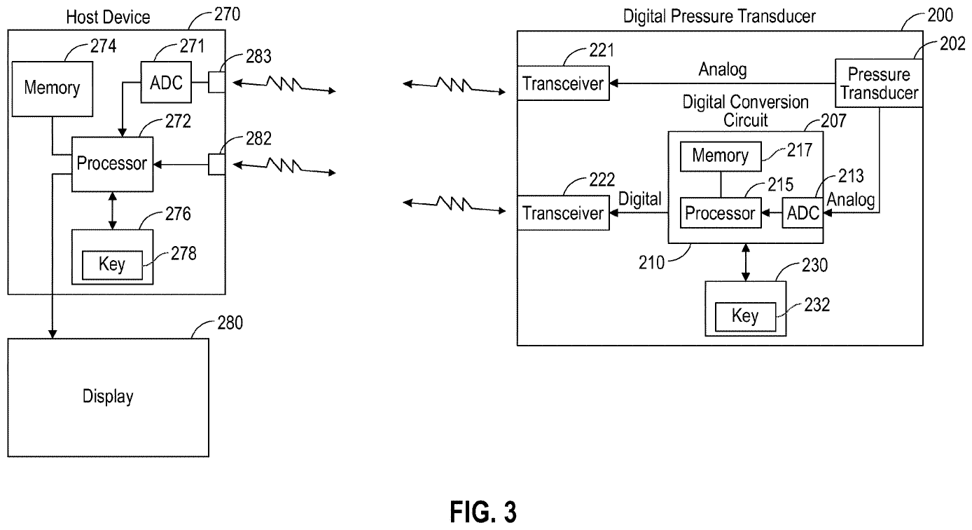

With additional reference to FIG. 3, in another optional example embodiment, the interface may be wireless transceivers 221 and 222 that wirelessly connect over the air to transceivers 283 and 282 of host device 270. In this optional example, transceiver 222 wirelessly transmits digital blood pressure measurements of the patient to host device 270 and transmits and receives authentication data for the performing of authentication with host device 270, as will be described in more detail hereafter. In one example, digital pressure transducer 200 and host device 270 wirelessly determine the authenticity of each other to start communication and thereafter the digital pressure transducer 200 wirelessly transmits the digital blood pressure measurements of the patient to the host device 270. Additionally, in an analog implementation, wireless transceiver 221 may transmit analog pressure signals from analog pressure transducer 202.

Continuing with this optional example embodiment, as shown in FIG. 3, the digital pressure transducer 200 includes: an analog pressure transducer 202, a digital conversion circuit 207, and a protected memory 230. The protected memory 230 stores a secret key 232. The digital conversion circuit 207 includes: an analog to digital converter (ADC) 213, a processor 215, and a memory 217. As has been described, the analog pressure transducer 202 measures an analog pressure of a fluid pressure from the fluid of the tube from the measurement site. The analog pressure signal is transmitted to ADC 213 to convert the analog pressure measurement from the analog pressure transducer 202 to a digital pressure measurement. Processor 215 is configured to convert the digital pressure measurement to a digital blood pressure measurement that is correlated to the blood pressure of the patient and to transmit the digital blood pressure measurement of the patient to the host device 270 through wireless transceiver 222 over the air to be received by wireless transceiver 282 of host device 270. Also, processor 210 may utilize the secret key 232 for authentication purposes that can be performed wirelessly via the transceivers 222 and 282, as will be described in more detail hereafter. As has been described, in one example, digital pressure transducer 200 and host device 270 wirelessly determine the authenticity of each other to start communication and thereafter the digital pressure transducer 200 wirelessly transmits the digital blood pressure measurements of the patient to the host device 270 over the air. Additionally, in some optional example embodiments, the analog pressure signal from the pressure transducer 202 may be directly connected wirelessly over the air to host device 270 via transceivers 221 and 283.

In the optional example embodiment, where the digital pressure transducer 200 is connected over the air wirelessly to the host device 270, various functions will be described. Digital blood pressure measurements of the patient from digital pressure transducer 240 are transmitted over the air wirelessly to the host device 270 from transceiver 222 of digital pressure transducer 200 to transceiver 282 of host device 270, as is authentication data for performing authentication of pressure transducer 340 with host device 270, as will be described in more detail hereafter. Also, in some optional example embodiments, digital pressure transducer 200 may also wirelessly transmit over the air the analog pressure signal from transceiver 221 to transceiver 283 of the host device. It should be appreciated that the optional examples may be utilized independently from one another or in combination with one another.

Continuing with this optional example embodiment of wireless connection, it should be appreciated that host device 270 includes: a processor 272, a memory 274, a secure memory 276 storing a secret key 278, an ADC 271, transceivers 282 and 283, and may be connected to a display 280. As has been described, in one example, digital pressure transducer 200 and host device 270 determine the authenticity of each other to start communication and thereafter the digital pressure transducer 200 transmits the digital blood pressure measurements of the patient to the host device 270 wirelessly. In this optional example embodiment, host device 270 under the control of processor 272 may receive the digital blood pressure measurements from the digital pressure transducer 200 corresponding the patient's blood pressure from transceiver 282 and may utilize them in monitoring diagnostic applications and/or other applications, and/or, may display the digital blood pressure measurements on display 280 (e.g., as a graphical depiction of blood pressure vs. time). It should be appreciated that a wide variety of other types of physiological data may be measured and displayed. Further, in some optional example embodiments, the host device 270 may convert the received analog pressure signals from digital pressure transducer 200 from transceiver 283 and via ADC 271 convert the analog pressure signal into digital pressure signals for processing by processor 272 such that the patient's digital blood pressure measurements can be analyzed and displayed, as previously described.

Also, in some optional example embodiments, the secret key 232 of secure memory 230 may be used with the digital pressure transducer 200 for authentication functions, as will be described in more detail hereafter. In particular, in one optional embodiment example, as will be described in more detail, digital pressure transducer 200 has authentication capabilities to wirelessly authenticate itself to host device 270, such that the host device 270 can be assured that it is receiving data from a legitimate digital pressure transducer. In this way, the digital pressure transducer 200 and the host device 270 wirelessly determine the authenticity of each other to start communication and thereafter the digital pressure transducer 200 wirelessly transmits the digital blood pressure measurements of the patient to the host device 270 through. It should be appreciated that the optional examples may be utilized independently from one another or in combination with one another. For example, the optional examples of utilizing wired cable connections or wireless connection may be utilized independently from one another or in combination with one another.

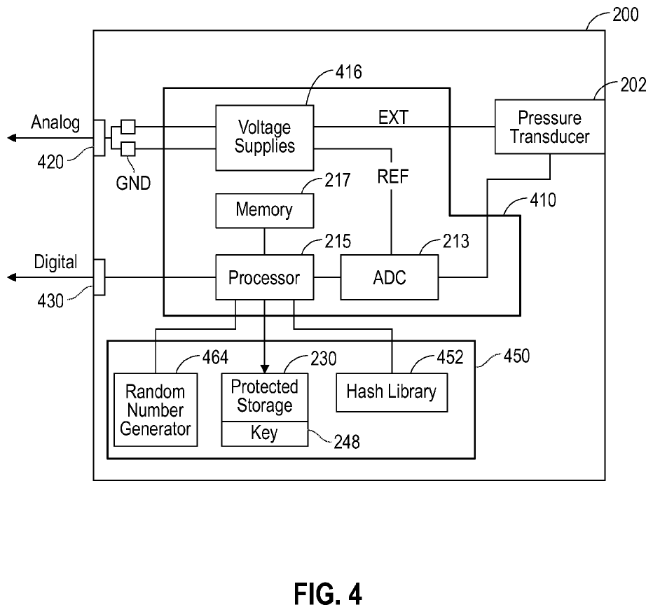

With additional reference to FIG. 4, an optional example embodiment of more detailed components of the digital pressure transducer 200 is described. As can be seen in FIG. 4, the digital pressure transducer 200 may comprise: an analog pressure transducer 202, a first circuit section 410, and a cybersecurity circuit section 450. The first circuit section may include: analog to digital converter (ADC) 213, processor 215, memory 217, and voltage supply 416. The cybersecurity circuit section 450 may include: a hash library 452, protected storage 230 that includes a secret key 248, and a random number generator 464.

As has been described, analog pressure transducer 202 measures an analog pressure of fluid pressure from the fluid of the tube from the measurement site to obtain an analog measurement of a patient's blood pressure. This analog pressure measurement can be inputted to voltage supply 416 along with a reference voltage form ADC 213 to produce an analog pressure measurement output 420. The analog pressure measurement output 420 can be outputted for analysis and display as a blood pressure measurement by host device 270 via a digital cable 240 (e.g., see FIG. 2 and associated description) or wirelessly (e.g., see FIG. 3 and associated description).

Further, as has been described, processor 215 may be configured to: perform authentication with the host device 270; convert the digital pressure measurement to a digital blood pressure measurement for the patient; and transmit the digital blood pressure measurement of the patient to the host device 270. In one example, digital pressure transducer 200 and host device 270 determine the authenticity of each other to start communication and thereafter the digital pressure transducer 200 transmits the digital blood pressure measurements of the patient to the host device 270 through an interface.

According to one optional example embodiment, as has been described, the analog pressure transducer 202 measures an analog pressure of a fluid pressure from the fluid of the tube from the measurement site. The analog pressure signal is transmitted to ADC 213 to convert the analog pressure measurement from the analog pressure transducer 202 to a digital pressure measurement. Processor 215 is configured to convert the digital pressure measurement to a digital blood pressure measurement 430 that is correlated to the blood pressure of the patient and to transmit the digital blood pressure measurement 430 of the patient to the host device 270. The digital blood pressure measurement 430 can be outputted for analysis and display as a blood pressure measurement by host device 270 via a digital cable 240 (e.g., see FIG. 2 and associated description) or wirelessly (e.g., see FIG. 3 and associated description).

Further, in one optional example embodiment, as shown in FIG. 4, processor 215 may interact with cybersecurity circuit section 450 that may include: a hash library 452, protected storage 230 that includes a secret key 248, and a random number generator 464-to implement authentication procedures.

As has been described, digital pressure transducer 200 and host device 270 determine the authenticity of each other to start communication and thereafter the digital pressure transducer 200 transmits the digital blood pressure measurements of the patient to the host device 270 through an interface. As one optional example embodiment, processor 215 may be configured to authenticate the digital pressure transducer 200 to host device 270 based upon a hash-based message authentication code (HMAC). As an example, in order to implement this authentication procedure, processor 215 may be configured to: command the random number 464 to generate a random number upon a request from the host device 270; generate a hash-based message authentication code (HMAC) based upon the random number and the secret key 248; and command the transmission of the HMAC to the host device as a digital output 430 to the host device. An appropriate hash function may be selected from the hash library 452. As example implementations, cybersecurity circuit section 450 may be a secure area of the integrated circuit that includes: hash library 452, protected storage 230 that includes a secret key 248, and a random number generator 464—to implement authentication procedures. As an example, the hash library 452 includes hash functions to be used in the HMAC generations to be discussed hereafter. The protected storage 230 may be a protected non-volatile memory (NVM) that includes one or more secret keys (only known by the digital pressure transducer and host device)-to implement authentication procedures. It should be appreciated that a secret key 248 should be unreadable (except by the digital pressure transducer itself) and non-extractable (e.g., through communication interface, x-ray, inspection methods, etc.). It should be appreciated that the optional examples may be utilized independently from one another or in combination with one another.

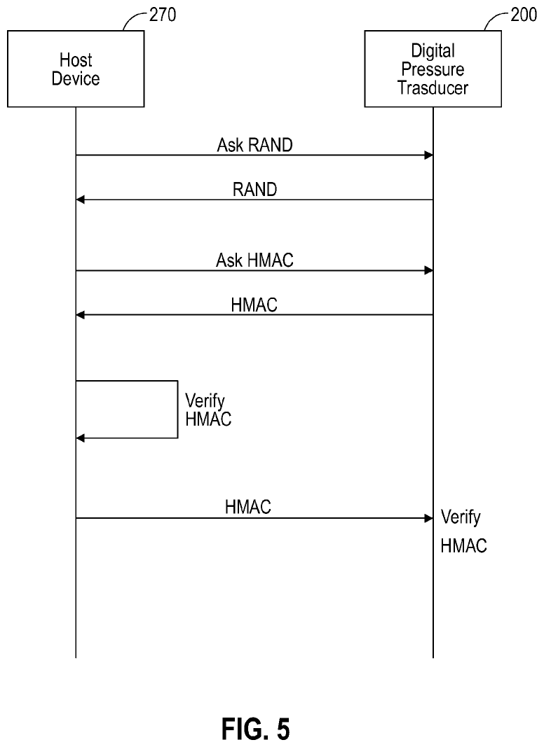

With additional brief reference to FIG. 5, an optional example embodiment of an authentication process for the digital pressure transducer will be described. As an example, this authentication process to authenticate the digital pressure transducer 200 may begin upon start-up and the initial connection between the pressure transducer 200 and the host device 270. In this example, host device 270 requests a random number (RAND) from the digital pressure transducer 200. Based upon this, processor 215 of digital pressure transducer 200 requests and obtains a random number from random number generator 464. Digital pressure transducer 200 then transmits the random number to host device 270. Next, host device 270 requests an HMAC from digital pressure transducer 200. Based upon the request, processor 215 of digital pressure transducer 200 obtains a secret key 248 from protected storage 230, a hash function from hash library 452 and calculates a HMAC based on the secret key 248, the hash function, and the random number. The processor 215 then commands the transmission of the HMAC to the host device 270.

As previously described, host device 270 similarly has a processor 272, protected storage 276, a secret key 278, etc., to perform authentication. Host device 270 under the control of its processer verifies the HMAC by calculating its own HMAC based on a hash function, the stored secret key, and the random number received by the digital pressure transducer. If the HMAC of the host device 270 matches the HMAC received from the digital pressure transducer 200, then the digital pressure transducer 200 is authenticated by the host device 270. In some additional examples, the digital pressure transducer 200 may verify the authenticity of the host device 270 in the same manner, e.g., asking for a random number, asking for an HMAC, calculating its own HMAC—and if the HMAC of the host device 270 matches the HMAC of the digital pressure transducer 200, then host device 270 is authenticated by the digital pressure transducer 200. It should be appreciated that the authentication data (RANDs, HMACs, etc.) for authentication can be transmitted and received via digital cable 240 (e.g., see FIG. 2 and associated description) or wirelessly (e.g., see FIG. 3 and associated description). Also, it should be appreciated the host device 270 may require authorization, at periodic intervals, from the pressure transducer to ensure that it is still a legitimate pressure transducer.

Additionally, in optional example embodiment, for authentication purposes, the digital cable 240 having a processor 242, a secret key 248 in secure storage 242 (see FIG. 2), may perform the same authentication process as implemented by the host device 270 in the same manner, as previously described, instead of the host device 270, for authenticating the digital pressure transducer 200. This optional example embodiment, may be used in scenarios where the host device cannot implement authentication functionality. It should be appreciated that the optional examples may be utilized independently from one another or in combination with one another.

Therefore, as previously described, digital pressure transducer 200 has authentication capabilities to authenticate itself to host device 270, such that the host device 270 can be assured that it is receiving data from a legitimate digital pressure transducer. In this way, the digital pressure transducer 200 and the host device 270 determine the authenticity of each other to start communication and thereafter the digital pressure transducer 200 can transmit the digital blood pressure measurements of the patient to the host device 270 through an interface, as previously described.

Therefore, as an example, upon start-up initiation, before blood pressure data is allowed to be transferred to the host device 270, pressure transducer 210 may be required to authenticate itself to the host device 270. If the pressure transducer 210 cannot authorize itself to the host device 270, as previously described, data transfer may not occur. In this way, the digital pressure transducer 210 and the host device 270 determine the authenticity of each other to start communication.

By utilizing the previously described authentication process, a digital pressure transducer 200 with authentication capability for use in a blood pressure monitoring system may be used that can authenticate itself to host device 270, such that the host device 270 can be assured that it is receiving data from a legitimate digital pressure transducer. Similarly, only host devices with the correct authentication features can access data from the digital pressure transducer. In this way, the digital pressure transducer and the host device can first determine the authenticity of each other to then start communication. This prevents pressure transducers without these authentication capabilities from communicating with a host device.

Also, after authentication, according to optional example embodiments, while host device 270 is receiving digital blood pressure readings from the digital pressure transducer 200, host device 200 can request validation of the integrity of the last N pressure readings. This can be utilized by host device 200 to ensure that digital blood pressure readings from the digital pressure transducer have not been interfered with or manipulated to very integrity. This can be done on demand by the host device 270. Also, host device 270 may perform this process on a predetermined schedule, randomly, or based upon predetermined conditions occurring.

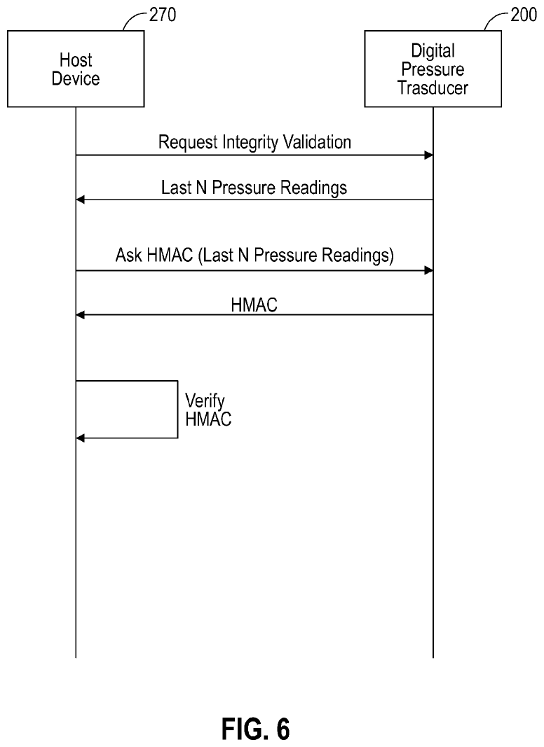

With additional brief reference to FIG. 6, an optional example embodiment of an integrity validation process will be described. First, host device 270 requests integrity validation from the digital pressure transducer 200 and, in particular, requests the last N digital pressure readings. Digital pressure transducer 200 then transmits the last N digital pressure readings. Next, host device 270 requests an HMAC based on the last N digital pressure readings from digital pressure transducer 200. Based upon this, processor 215 of digital pressure transducer 200 obtains a secret key 248 from protected storage 230, a hash function from hash library 452, and calculates a HMAC based on the secret key 248, the hash function, and the last N digital pressure readings. The processor 215 then commands the transmission of the HMAC to the host device 270.

As previously described, host device 270 similarly has a processor 272, protected storage 276, a secret key 278, etc., to perform integrity validation. Host device 270 under the control of its processer verifies the HMAC by calculating its own HMAC based on a hash function, the stored secret key, and last N digital pressure readings previously received by the digital pressure transducer 200. If the HMAC of the host device 270 matches the HMAC received from the digital pressure transducer 200, then the integrity of the digital pressure readings from the digital pressure transducer 200 are validated by the host device 270. It should be appreciated that the integrity validation data (pressure readings, HMACs, etc.) for integrity validation can be transmitted and received via the digital cable 240 (e.g., see FIG. 2 and associated description) or wirelessly (e.g., see FIG. 3 and associated description). Also, N pressure readings may be any suitable amount of digital pressure readings-10, 50, 100, 1000, etc. (e.g., any suitable amount that can be utilized in HMAC calculations in a suitable amount of time).

Additionally, in one optional example embodiment, for integrity validation purposes, the digital cable 240 having a processor 242, a secret key 248 in secure storage 242 (e.g., see FIG. 2), may perform the same integrity validation process as implemented by the host device 270, in the same manner, as previously described, instead of the host device 270. This optional example embodiment, may be used in scenarios where the host device cannot implement integrity validation functionality. It should be appreciated that the optional examples may be utilized independently from one another or in combination with one another. Also, it should be appreciated that the optional examples of authentication and integrity validation may be utilized independently from one another or in combination with one another.

By utilizing the previously described integrity validation process, a digital pressure transducer 200 with integrity validation capability for use in a blood pressure monitoring system can validate the integrity of its digital blood pressure readings to the host device 270, such that the host device 270 can be assured that the digital blood pressure readings that it is receiving from the digital pressure transducer 200 have not been interfered with, manipulated, or tampered with. This can be done on demand by the host device 270. For example, host device 270 may perform this process on a predetermined schedule, randomly, or based upon predetermined conditions occurring.

As previously described with references to FIGS. 1-4, the blood pressure monitoring system may include a measurement site 108, a tube 109, and a host device 270, in which, the tube 109 is coupled between the measurement site 108 of the patient 102 and the digital pressure transducer 200, and, in which, the tube 109 contains a fluid for pressure measurement from the measurement site 108. Digital pressure transducer 200 may comprise: an analog pressure transducer 202 to measure an analog pressure of a fluid pressure from the fluid of the tube from the measurement site; an analog to digital converter (ADC) 213 to convert the analog pressure measurement from the analog pressure transducer to a digital pressure measurement; an interface to connect to the host device 270; and a processor 215. Processor 215 may be configured to: convert the digital pressure measurement to a digital blood pressure measurement for the patient; transmit the digital blood pressure measurement of the patient to the host device 270; and perform integrity validation with the host device 270. Further, as has been described, host device 270 under the control of processor 272 may receive the digital blood pressure measurements from the digital pressure transducer 200 corresponding the patient's blood pressure and may utilize them in monitoring diagnostic applications and/or other applications, and/or, may display the digital blood pressure measurements on display 280.

In one optional example, digital pressure transducer 200 may be connected to host device 270 by a digital cable 240, in which, the digital cable 240 transmits integrity validation data for the performing of integrity validation with the host device and the digital blood pressure measurement of the patient to the host device 270. In one optional example, the interface is a wireless transceiver 222 that wirelessly connects over the air to a transceiver of the host device, and the transceiver wirelessly transmits and receives integrity validation data for the performing of integrity validation with the host device 270 and wirelessly transmits the digital blood pressure measurement of the patient to the host device 270. In one optional example, the interface is a digital cable 240, and the digital cable 240 is connected to the host device 270, and the digital cable performs integrity validation with the digital pressure transducer 200, and the digital cable 240 transmits the digital blood pressure measurement of the patient to the host device.

Further, in one optional example embodiment, as has been described, digital pressure transducer 200 may include protected storage 230 storing a secret key 248, and processor 215 of digital pressure transducer 200 may be configured to perform integrity validation of a predefined number of digital blood pressure measurements transmitted to the host device 270 based upon a hash-based message authentication code (HMAC). In one example to perform integrity validation with the host device 270, the processor is further configured to: obtain the predefined number of digital blood pressure measurements transmitted to the host device; generate a hash-based message authentication code (HMAC) based upon predefined number of digital blood pressure measurements and the secret key; and command the interface to transmit the HMAC to the host device. It should be appreciated that the optional examples may be utilized independently from one another or in combination with one another.

As previously described with references to FIGS. 1-4, the blood pressure monitoring system may include a measurement site 108, a tube 109, and a host device 270, in which, the tube 109 is coupled between the measurement site 108 of the patient 102 and the digital pressure transducer 200, and, in which, the tube 109 contains a fluid for pressure measurement from the measurement site 108. Digital pressure transducer 200 for use in measuring the blood pressure of a patient and providing authentication may comprise: an analog pressure transducer 202 to measure an analog pressure of a fluid pressure from the fluid of the tube from the measurement site; an analog to digital converter (ADC) 213 to convert the analog pressure measurement from the analog pressure transducer to a digital pressure measurement; an interface to connect to the host device 270; and a processor 215. Processor 215 may be configured to: perform authentication with the host device 270; convert the digital pressure measurement to a digital blood pressure measurement for the patient; and transmit the digital blood pressure measurement of the patient to the host device 270. In one example, digital pressure transducer 200 and host device 270 determine the authenticity of each other to start communication and thereafter the digital pressure transducer 200 transmits the digital blood pressure measurements of the patient to the host device 270 through an interface. Further, as has been described, host device 270 under the control of processor 272 may receive the digital blood pressure measurements from the digital pressure transducer 200 corresponding the patient's blood pressure and may utilize them in monitoring diagnostic applications and/or other applications, and/or, may display the digital blood pressure measurements on display 280.

In one optional example embodiment, digital pressure transducer 200 may be connected host device 270 by a digital cable 240, in which, the digital cable 240 transmits authentication data for the performing authentication with the host device and the digital blood pressure measurement of the patient to the host device 270. In one optional example, the interface is a wireless transceiver 222 that wirelessly connects over the air to a transceiver of the host device, and the transceiver wirelessly transmits and receives authentication data for the performing authentication with the host device 270 and wirelessly transmits the digital blood pressure measurement of the patient to the host device 270. In one optional example, the interface is a digital cable 240, and the digital cable 240 is connected to the host device 270, and the digital cable performs authentication with the digital pressure transducer 200, and the digital cable 240 transmits the digital blood pressure measurement of the patient to the host device.

Further, in one optional example embodiment, as has been described, digital pressure transducer 200 may include protected storage 230 storing a secret key 248, a random number generator 464, and processor 215 of digital pressure transducer 200 may be configured to authenticate digital pressure transducer 200 to host device 270 based upon a hash-based message authentication code (HMAC). In one example to perform authentication with host device 270, processor 215 is further configured to: command random number generator 464 to generate a random number upon a request from the host device; generate a hash-based message authentication code (HMAC) based upon the random number and the secret key 248; and command the interface to transmit the HMAC to host device 270.

It should be appreciated that the various previously described optional example implementations throughout the disclosure may be utilized independently from one another or in combination with one another. For example, it should be appreciated that aspects of the optional examples of FIGS. 2 and 3 as to the use of a digital cable and/or wireless communication between the digital pressure transducer and the host device may be used independently of one another, or in combination with one another. Further, it should be appreciated that aspects of the optional examples of FIGS. 5 and 6, regarding authentication processes and integrity validation processes may be used independently of one another, or in combination with one another. Accordingly, it should be appreciated that a wide variety of the previously described optional examples may be utilized independently from one another or in combination with one or more of them, in a suitable configuration.

By utilizing the previously described authentication process, a digital pressure transducer 200 with authentication capability for use in a blood pressure monitoring system may be used that can authenticate itself to host device 270, such that the host device 270 can be assured that it is receiving data from a legitimate digital pressure transducer. Further, by utilizing the previously described integrity validation process, a digital pressure transducer 200 with integrity validation capability for use in a blood pressure monitoring system can validate the integrity of its digital blood pressure readings to the host device 270, such that the host device 270 can be assured that the digital blood pressure readings that it is receiving from the digital pressure transducer 200 have not been interfered with, manipulated, or tampered with.

It should be appreciated that although the digital pressure transducer and the host device have been described as measuring, analyzing, and reporting blood pressure related measurement values in combination with authentication and integrity validation features, that these features may also be applied to other hemodynamic values-carbon monoxide (CO), systemic vascular resistance (SVR), stroke volume variation (SVV), etc. As is well understood, patient monitors may employ sophisticated algorithms to derive volumetric and hemodynamic parameters from the pressure signal.

It should be appreciated that aspects of the invention previously described may be implemented in conjunction with the execution of instructions or code by processors, circuitry, controllers, control circuitry, etc. (e.g., processors 215, 272, etc.). As an example, processors may operate under the control of a program, algorithm, code, routine, or the execution of instructions to execute methods or processes (e.g., processes in FIGS. 5 and 6) in accordance with embodiments of the invention previously described. For example, such a program may be implemented in firmware or software (e.g., stored in memory and/or other locations) (e.g., stored in memories 217, 274) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, non-transitory computer readable medium, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the invention. Various modifications to these embodiments will be readily apparent to those skilled in the art, and the generic principles defined herein may be applied to other embodiments without departing from the spirit or scope of the invention. Thus, the invention is not intended to be limited to the embodiments shown herein but is to be accorded the widest scope consistent with the principles and novel features disclosed herein.

The disclosure also includes the following clauses:
1. A digital pressure transducer for use in a blood pressure monitoring system to measure the blood pressure of a patient and to provide integrity validation, the blood pressure monitoring system including a measurement site, a tube, and a host device, the tube coupled between the measurement site of the patient and the digital pressure transducer, and the tube containing a fluid for pressure measurement from the measurement site, the digital pressure transducer comprising:
   an analog pressure transducer to measure an analog pressure of a fluid pressure from the fluid of the tube from the measurement site;
   an analog to digital converter (ADC) to convert the analog pressure measurement to a digital pressure measurement; an interface to connect to the host device; and
   a processor configured to:
      convert the digital pressure measurement to a digital blood pressure measurement for the patient;
      transmit the digital blood pressure measurement of the patient to the host device; and
perform integrity validation with the host device.
2. The digital pressure transducer of claim 1, wherein, the processor is further configured to perform integrity validation for a predefined number of digital blood pressure measurements transmitted to the host device based upon a hash-based message authentication code (HMAC).
3. The digital pressure transducer of any of the claims 1-2, wherein, the digital pressure transducer further comprises a secret key.
4. The digital pressure transducer of any of the claims 1-3, wherein, to perform integrity validation with the host device, the processor is further configured to:
   obtain a predefined number of digital blood pressure measurements transmitted to the host device;
   generate a hash-based message authentication code (HMAC) based upon the predefined number of digital blood pressure measurements and a secret key; and
   command the interface to transmit the HMAC to the host device.
5. The digital pressure transducer of any of the claims 1-4, wherein, the interface is a digital cable, and the digital cable is connected to the host device, and the digital cable transmits integrity validation data for the performing of integrity validation with the host device and the digital blood pressure measurement of the patient to the host device.
6. The digital pressure transducer of any of the claims 1-4, wherein, the interface is a wireless transceiver that wirelessly connects over the air to a transceiver of the host device, and the transceiver wirelessly transmits and receives integrity validation data for the performing of integrity validation with the host device and wirelessly transmits the digital blood pressure measurement of the patient to the host device.
7. The digital pressure transducer of any of the claims 1-6, wherein, the digital pressure transducer is a disposable pressure transducer.
8. A non-transitory computer-readable medium comprising code which, when executed by a processor of digital pressure transducer for use in a blood pressure monitoring system to measure the blood pressure of a patient and to provide integrity validation, the blood pressure monitoring system including a measurement site, a tube, and a host device, the tube coupled between the measurement site of the patient and the digital pressure transducer, and the tube containing a fluid for pressure measurement from the measurement site, causes the processor to perform operations comprising:
   converting a digital pressure measurement to a digital blood pressure measurement for the patient, wherein the digital pressure measurement is received from an analog to digital converter (ADC) that converts an analog pressure measurement of a fluid pressure from the fluid of the tube from the measurement site to the digital pressure measurement;
   transmitting the digital blood pressure measurement of the patient to the host device; and
   performing integrity validation with the host device.
9. The non-transitory computer-readable medium of claim 8, wherein, to perform integrity validation with the host device, the processor further performs the operations of performing integrity validation for a predefined number of digital blood pressure measurements transmitted to the host device based upon a hash-based message authentication code (HMAC).
10. The non-transitory computer-readable medium of any of the claims 8-9, wherein, the digital pressure transducer further comprises a secret key.
11. The non-transitory computer-readable medium of any of the claims 8-10, wherein, to perform integrity validation with the host device, the processor further performs the operations of:
   obtaining a predefined number of digital blood pressure measurements transmitted to the host device;
   generating a hash-based message authentication code (HMAC) based upon the predefined number of digital blood pressure measurements and a secret key; and
   commanding the interface to transmit the HMAC to the host device.
12. The non-transitory computer-readable medium of any of the claims 8-11, wherein, the digital pressure transducer is a disposable pressure transducer.
13. A blood pressure monitoring system to measure the blood pressure of a patient from a measuring site and to provide integrity validation, the blood pressure monitoring system comprising:
   a host device;
   a tube coupled between the measurement site of the patient and a digital pressure transducer, the tube containing a fluid for pressure measurement from the measurement site, wherein the digital pressure transducer includes:
      an analog pressure transducer to measure an analog pressure of a fluid pressure from the fluid of the tube from the measurement site;
an analog to digital converter (ADC) to convert the analog pressure measurement to a digital pressure measurement;
   an interface to connect to the host device; and
   a processor configured to:
      convert the digital pressure measurement to a digital blood pressure measurement for the patient;
      transmit the digital blood pressure measurement of the patient to the host device; and
perform integrity validation with the host device.
14. The blood pressure monitoring system of claim 13, wherein, the processor is further configured to perform integrity validation for a predefined number of digital blood pressure measurements transmitted to the host device based upon a hash-based message authentication code (HMAC).
15. The blood pressure monitoring system of any of the claims 13-14, wherein, the digital pressure transducer further comprises a secret key.
16. The blood pressure monitoring system of any of the claims 13-15, wherein, to perform integrity validation with the host device, the processor is further configured to:
   obtain a predefined number of digital blood pressure measurements transmitted to the host device;
   generate a hash-based message authentication code (HMAC) based upon the predefined number of digital blood pressure measurements and a secret key; and
   command the interface to transmit the HMAC to the host device.
17. The blood pressure monitoring system of any of the claims 13-16, wherein, the interface is a digital cable, and the digital cable is connected to the host device, and the digital cable transmits integrity validation data for the performing of integrity validation with the host device and the digital blood pressure measurement of the patient to the host device.
18. The blood pressure monitoring system of any of the claims 13-16, wherein, the interface is a wireless transceiver that wirelessly connects over the air to a transceiver of the host device, and the transceiver wirelessly transmits and receives integrity validation data for the performing of integrity validation with the host device and wirelessly transmits the digital blood pressure measurement of the patient to the host device.
19. The blood pressure monitoring system of any of the claims 13-16, wherein, the interface is a digital cable, and the digital cable is connected to the host device, and the digital cable performs integrity validation with the digital pressure transducer, and the digital cable transmits the digital blood pressure measurement of the patient to the host device.
20. The blood pressure monitoring system of any of the claims 13-19, wherein, the digital pressure transducer is a disposable pressure transducer.
21. A digital pressure transducer for use in a blood pressure monitoring system to measure the blood pressure of a patient and to provide integrity validation, the blood pressure monitoring system including a measurement site, a tube, and a host device, the tube coupled between the measurement site of the patient and the digital pressure transducer, and the tube containing a fluid for pressure measurement from the measurement site, the digital pressure transducer comprising:
   an analog pressure transducer to measure an analog pressure of a fluid pressure from the fluid of the tube from the measurement site;
   an analog to digital converter (ADC) to convert the analog pressure measurement to a digital pressure measurement;
   a secret key;
   an interface to connect to the host device; and
   a processor configured to perform integrity validation with the host device by performing operations including:
      obtaining a predefined number of digital blood pressure measurements previously transmitted to the host device;
      generating a hash-based message authentication code (HMAC) based upon the predefined number of digital blood pressure measurements and the secret key;
      commanding the interface to transmit the HMAC to the host device such that the host device is capable of integrity validation of the predefined number of digital blood pressure measurements;
      converting subsequent digital pressure measurements to digital blood pressure measurements for the patient; and
      transmitting the subsequent digital blood pressure measurements of the patient to the host device.

1. A digital pressure transducer for use in a blood pressure monitoring system to measure the blood pressure of a patient and to provide integrity validation, the blood pressure monitoring system including a measurement site, a tube, and a host device, the tube coupled between the measurement site of the patient and the digital pressure transducer, and the tube containing a fluid for pressure measurement from the measurement site, the digital pressure transducer comprising:
an analog pressure transducer to measure an analog pressure of a fluid pressure from the fluid of the tube from the measurement site;
an analog to digital converter (ADC) to convert the analog pressure measurement to a digital pressure measurement;
an interface to connect to the host device; and
a processor configured to:
convert the digital pressure measurement to a digital blood pressure measurement for the patient;
transmit the digital blood pressure measurement of the patient to the host device; and perform integrity validation with the host device.

2. The digital pressure transducer of claim 1, wherein, the processor is further configured to perform integrity validation for a predefined number of digital blood pressure measurements transmitted to the host device based upon a hash-based message authentication code (HMAC).

3. The digital pressure transducer of claim 1, wherein, the digital pressure transducer further comprises a secret key.

4. The digital pressure transducer of claim 1, wherein, to perform integrity validation with the host device, the processor is further configured to:
obtain a predefined number of digital blood pressure measurements transmitted to the host device; generate a hash-based message authentication code (HMAC) based upon the predefined number of digital blood pressure measurements and a secret key; and
command the interface to transmit the HMAC to the host device.

5. The digital pressure transducer of claim 1, wherein, the interface is a digital cable, and the digital cable is connected to the host device, and the digital cable transmits integrity validation data for the performing of integrity validation with the host device and the digital blood pressure measurement of the patient to the host device.

6. The digital pressure transducer of claim 1, wherein, the interface is a wireless transceiver that wirelessly connects over the air to a transceiver of the host device, and the transceiver wirelessly transmits and receives integrity validation data for the performing of integrity validation with the host device and wirelessly transmits the digital blood pressure measurement of the patient to the host device.

7. The digital pressure transducer of claim 1, wherein, the digital pressure transducer is a disposable pressure transducer.

8. A non-transitory computer-readable medium comprising code which, when executed by a processor of digital pressure transducer for use in a blood pressure monitoring system to measure the blood pressure of a patient and to provide integrity validation, the blood pressure monitoring system including a measurement site, a tube, and a host device, the tube coupled between the measurement site of the patient and the digital pressure transducer, and the tube containing a fluid for pressure measurement from the measurement site, causes the processor to perform operations comprising:
converting a digital pressure measurement to a digital blood pressure measurement for the patient, wherein the digital pressure measurement is received from an analog to digital converter (ADC) that converts an analog pressure measurement of a fluid pressure from the fluid of the tube from the measurement site to the digital pressure measurement;
transmitting the digital blood pressure measurement of the patient to the host device; and
performing integrity validation with the host device.

9. The non-transitory computer-readable medium of claim 8, wherein, to perform integrity validation with the host device, the processor further performs the operations of performing integrity validation for a predefined number of digital blood pressure measurements transmitted to the host device based upon a hashbased message authentication code (HMAC).

10. The non-transitory computer-readable medium of claim 8, wherein, the digital pressure transducer further comprises a secret key.

11. The non-transitory computer-readable medium of claim 8, wherein, to perform integrity validation with the host device, the processor further performs the operations of:
obtaining a predefined number of digital blood pressure measurements transmitted to the host device;
generating a hash-based message authentication code (HMAC) based upon the predefined number of digital blood pressure measurements and a secret key; and
commanding the interface to transmit the HMAC to the host device.

12. The non-transitory computer-readable medium of claim 8, wherein, the digital pressure transducer is a disposable pressure transducer.

13. A blood pressure monitoring system to measure the blood pressure of a patient from a measuring site and to provide integrity validation, the blood pressure monitoring system comprising:
a host device;
a tube coupled between the measurement site of the patient and a digital pressure transducer, the tube containing a fluid for pressure measurement from the measurement site, wherein the digital pressure transducer includes:
an analog pressure transducer to measure an analog pressure of a fluid pressure from the fluid of the tube from the measurement site;
an analog to digital converter (ADC) to convert the analog pressure measurement to a digital pressure measurement;
an interface to connect to the host device; and
a processor configured to:
convert the digital pressure measurement to a digital blood pressure measurement for the patient;
transmit the digital blood pressure measurement of the patient to the host device; and perform integrity validation with the host device.

14. The blood pressure monitoring system of claim 13, wherein, the processor is further configured to perform integrity validation for a predefined number of digital blood pressure measurements transmitted to the host device based upon a hash-based message authentication code (HMAC).

15. The blood pressure monitoring system of claim 13, wherein, the digital pressure transducer further comprises a secret key.

16. The blood pressure monitoring system of claim 13, wherein, to perform integrity validation with the host device, the processor is further configured to:
obtain a predefined number of digital blood pressure measurements transmitted to the host device; generate a hash-based message authentication code (HMAC) based upon the predefined number of digital blood pressure measurements and a secret key; and
command the interface to transmit the HMAC to the host device.

17. The blood pressure monitoring system of claim 13, wherein, the interface is a digital cable, and the digital cable is connected to the host device, and the digital cable transmits integrity validation data for the performing of integrity validation with the host device and the digital blood pressure measurement of the patient to the host device.

18. The blood pressure monitoring system of claim 13, wherein, the interface is a wireless transceiver that wirelessly connects over the air to a transceiver of the host device, and the transceiver wirelessly transmits and receives integrity validation data for the performing of integrity validation with the host device and wirelessly transmits the digital blood pressure measurement of the patient to the host device.

19. The blood pressure monitoring system of claim 13, wherein, the interface is a digital cable, and the digital cable is connected to the host device, and the digital cable performs integrity validation with the digital pressure transducer, and the digital cable transmits the digital blood pressure measurement of the patient to the host device.

20. The blood pressure monitoring system of claim 13, wherein, the digital pressure transducer is a disposable pressure transducer.

21. A digital pressure transducer for use in a blood pressure monitoring system to measure the blood pressure of a patient and to provide integrity validation, the blood pressure monitoring system including a measurement site, a tube, and a host device, the tube coupled between the measurement site of the patient and the digital pressure transducer, and the tube containing a fluid for pressure measurement from the measurement site, the digital pressure transducer comprising:
an analog pressure transducer to measure an analog pressure of a fluid pressure from the fluid of the tube from the measurement site;
an analog to digital converter (ADC) to convert the analog pressure measurement to a digital pressure measurement;
a secret key;
an interface to connect to the host device; and
a processor configured to perform integrity validation with the host device by performing operations including:
obtaining a predefined number of digital blood pressure measurements previously transmitted to the host device;
generating a hash-based message authentication code (HMAC) based upon the predefined number of digital blood pressure measurements and the secret key;
commanding the interface to transmit the HMAC to the host device such that the host device is capable of integrity validation of the predefined number of digital blood pressure measurements;
converting subsequent digital pressure measurements to digital blood pressure measurements for the patient; and
transmitting the subsequent digital blood pressure measurements of the patient to the host device.
